(19) United States
(12) Patent Application Publication
(10) Pub. No.: US 2024/0239848 A1
(43) Pub. Date: Jul. 18, 2024

(54) HOLOTHURIAN-DERIVED ACTIVE PEPTIDE WITH IMMUNE ACTIVITY, PREPARATION METHOD AND APPLICATION THEREOF

(71) Applicant: BOHAI UNIVERSITY, Jinzhou (CN)

(72) Inventors: Xuepeng LI, Jinzhou (CN); Junnan YANG, Jinzhou (CN); Jianrong LI, Jinzhou (CN); Jinxiang WANG, Jinzhou (CN); Fangchao CUI, Jinzhou (CN); Tingting LI, Jinzhou (CN); Yingmei LI, Jinzhou (CN); Qing YANG, Jinzhou (CN); Zhengpeng WEI, Jinzhou (CN)

(21) Appl. No.: 18/396,269

(22) Filed: Dec. 26, 2023

(30) Foreign Application Priority Data
Jan. 18, 2023 (CN) 202310062959.7

(51) Int. Cl. C07K 14/435; A61P 37/04

(52) U.S. Cl. CPC C07K 14/43504; A61P 37/04

(57) ABSTRACT

A holothurian-derived active peptide with immune activity, a preparation method and an application thereof are provided in the present disclosure, belonging to the field of biotechnology. The amino acid sequence of the holothurian-derived active peptide is shown in SEQ ID NO.1. The amino acid sequence of the holothurian-derived immunocompetent peptide is obtained using separation and purification techniques and mass spectrometry identification techniques with the help of the holothurian protein database, and the interaction of the immunocompetent peptide with the membrane recognition receptor (toll-like receptor 2) is explored by molecular docking technique and multiple rounds of screening, and the in vitro immunological activity of the peptide is verified by solid-phase synthesis and RAW264.7 cell model.

### CROSS-REFERENCE TO RELATED APPLICATIONS

This application claims priority to Chinese Patent Application No. 202310062959.7, filed on Jan. 18, 2023, the contents of which are hereby incorporated by reference.

### INCORPORATION BY REFERENCE STATEMENT

This statement, made under Rules 77(b)(5)(ii) and any other applicable rule incorporates into the present specification of an XML file for a “Sequence Listing XML” (see Rule 831(a)), submitted via the USPTO patent electronic filing system or on one or more read-only optical discs (see Rule 1.52(e)(8)), identifying the names of each file, the date of creation of each file, and the size of each file in bytes as follows:
File name: sequence_347-108_13308
Creation date: Nov. 22, 2023
Byte size: 12,148

### TECHNICAL FIELD

The present disclosure relates to the field of biotechnology, and in particular to a holothurian-derived active peptide with immune activity, and a preparation method and an application thereof.

### BACKGROUND

People in modern society are facing an increasingly fast pace of life and greater work pressure, their bodies are often in a state of stress, resulting in low immunity, which is the root cause of diseases. Studies have shown that immunocompetent peptides are capable of enhancing immunity and protecting the organism from pathogen invasion by inhibiting oxidative stress. Based on the existing technology, a lot of research has been carried out on immunocompetent peptides from food-borne animal and plant proteins, with marine protein-derived immunocompetent peptides receiving extensive attention by virtue of their high safety and structural stability. Accordingly, marine bio-derived immunocompetent peptides have become one of the hot topics of concern at present.

As a species of invertebrate, holothurian belongs to the phylum Echinodermata and the class Holothuroidea, with more than 1000 years of commercial fishing history. Modern research has proved that holothurian is rich in protein, essential amino acids, various trace elements and vitamins, with more than 50 kinds of nutrients beneficial to the organism, as well as various biologically active substances that are effective in antioxidant, anti-tumour, anti-fatigue, lowering blood pressure, lowering blood lipids and modulating immunity. At present, most of the studies on active peptides of holothurian focus on the properties of antioxidant, anti-fatigue, etc., with relatively fewer studies related to immune-activated peptides. The screening and development of new immunocompetent peptides from holothurian is of great significance for enriching the variety of immunocompetent peptides of marine protein sources, preventing diseases, enhancing body immunity, etc., as well as developing deep processing of marine protein resources.

### SUMMARY

The objective of the present disclosure is to provide a holothurian-derived active peptide with immune activity, and a preparation method and an application thereof, so as to solve the problems existing in the prior art. The present disclosure provides an immunocompetent peptide with good immune activity and no biological toxicity, it activates the macrophages of the body to develop an immune response with anti-inflammatory effect, and it is stable in the intestinal tract, with great significance to the deep processing of marine protein resources.

In order to achieve the above objectives, the present disclosure provides the following schemes.

The present disclosure provides a holothurian-derived active peptide with immune activity, where an amino acid sequence of the holothurian-derived active peptide is shown in SEQ ID NO.1.

The present disclosure also provides a preparation of immunocompetent peptide, including the holothurian-derived active peptide.

The present disclosure also provides a preparation method of the holothurian-derived active peptide, including: using holothurian enzymatic hydrolysate as raw material, carrying out ultrafiltration and gel chromatography for separation and purification, screening out components of strongest immunological activity, then identifying by secondary mass spectrometry, and screening out the holothurian-derived active peptide by comparing with a holothurian protein database.

Optionally, a preparation method of the holothurian enzymatic hydrolysate includes: mixing holothurian with water to obtain a mixture, adding neutral protease into the mixture for enzymolysis, carrying out water bath heating to inactivate enzymes at an end of the enzymolysis, and centrifuging twice, extracting a supernatant to obtain the holothurian enzymatic hydrolysate.

Optionally, a material-liquid ratio of holothurian to water is 1 gram (g):12 milliliters (mL).

Optionally, an addition amount of the neutral protease is 0.6% of a mass of the mixture.

Optionally, a duration of the enzymolysis is 300 minutes (min), a temperature is 50 degrees Celsius (° C.) and a pH is 7.0.

Optionally, a temperature of the water bath heating is 100° C. and a duration is 10 min.

The present disclosure also provides an application of the holothurian-derived active peptide or the preparation of immunocompetent peptide in preparing products for improving immunity.

Optionally, the products include medicines.

The present disclosure uses separation and purification techniques and mass spectrometry identification techniques to obtain the amino acid sequence of the holothurian-derived immunocompetent peptide with the help of the holothurian protein database, and carries out multiple rounds of screening by determining the influences of the active peptides on the survival rate, cell phagocytosis, release of NO, and cytokine (Interleukin 1β (IL-1β), Tumor necrosis factor alpha (TNF-α)) secretion from the macrophage RAW264.7, as well as properties of the peptides such as the amino acid composition and biotoxicity; the interaction of the immunocompetent peptide with the membrane recognition receptor (toll-like receptor 2, abbreviated as TLR-2) is explored by the molecular docking technique, and the in vitro immunological activity of the peptide is verified by solid-phase synthesis and RAW264.7 cell model; the results show that the novel immunocompetent peptide obtained from the screening of the present disclosure has in vitro immunological activity without biotoxicity, which activates, yet not excessively activates, the macrophages of the organism to develop an immune response and anti-inflammatory effect; it is stable in the intestinal tract and enables the organism to be in a healthy and stable state. Moreover, the new active peptide sequences obtained by the present disclosure provide new resources for functional food development.

### BRIEF DESCRIPTION OF THE DRAWINGS

For a clearer description of the technical schemes in the embodiments or prior art of the present disclosure, the accompanying drawings to be used in the embodiments are briefly described hereinafter, and it is obvious that the accompanying drawings in the description hereinafter are only some of the embodiments of the present disclosure, and that for the person of ordinary skill in the field, other accompanying drawings are available on the basis of the accompanying drawings without any creative labour.

FIG. 1A is a molecular docking diagram of IIENAVQ and toll-like receptor 2 (TLR-2).

FIG. 1B is a molecular docking diagram of FAGDDAPRA and TLR-2.

FIG. 1C is a molecular docking diagram of KSYELP and TLR-2.

FIG. 2 illustrates influences of synthetic peptide on the macrophage RAW264.7 in terms of a survival rate.

FIG. 3 shows influences of the synthetic peptide activating macrophage RAW264.7 to release NO.

FIG. 4A shows influences of IIENAVQ activation and lipopolysaccharide (LPS) induction on a secretion of cytokine Interleukin 1β (IL-1β) of the macrophage RAW264.7.

FIG. 4B shows influences of IIENAVQ activation and LPS induction on a secretion of cytokine Tumor necrosis factor alpha (TNF-α) of the macrophage RAW264.7.

FIG. 5 is a process illustrating a preparation method of a holothurian-derived active peptide of the present disclosure.

FIG. 6 shows a process of a preparation method of holothurian enzymatic hydrolysate provided by the present disclosure.

### DETAILED DESCRIPTION OF THE EMBODIMENTS

Various exemplary embodiments of the present disclosure are now described in detail, and this detailed description should not be considered as a limitation of the present disclosure, but should be understood as a more detailed description of certain aspects, features and embodiments of the present disclosure.

It is to be understood that the terms described in the present disclosure are intended only to describe particular embodiments and are not intended to limit the present disclosure. Further, for the range of values in the present disclosure, it should be understood that each intermediate value between the upper and lower limits of the range is also specifically disclosed. Each smaller range between any stated value or intermediate value within a stated range and any other stated value or intermediate value within the range is also included within the present disclosure. The upper and lower limits of these smaller ranges may be independently included or excluded from the range.

Unless otherwise specified, all technical and scientific terms used herein have the same meaning as commonly understood by one of ordinary skill in the art to which this disclosure relates. Although the present disclosure only describes the preferred methods and materials, any methods and materials similar or equivalent to those described herein can also be used in the practice or testing of the present disclosure. All documents mentioned in this specification are incorporated by reference to disclose and describe methods and/or materials related to the documents. In case of conflict with any incorporated document, the contents of this specification shall prevail.

It is obvious to those skilled in the art that many improvements and changes are available to the specific embodiments of the present disclosure without departing from the scope or spirit of the present disclosure. Other embodiments will be apparent to the skilled person from the description of the disclosure. The specification and embodiments of this disclosure are only exemplary.

The terms “including”, “comprising”, “having” and “containing” used in this specification are all open terms, which means including but not limited to.

Instruments and chemicals used in the embodiments of the present disclosure are as follows:

AKTA Purifier 100 protein purifier, Q Exactive™ Mass Spectrometer (Thermo Fisher Scientific, MA, USA) of EASY-nanoLC 1200 combined with electrospray ionization (ESI); RAW264.7 mouse mononuclear macrophages, purchased from the Center for Excellence in Molecular Cell Science of China Academy of Sciences; lipopolysaccharide (LPS), purchased from Merck Biotechnology (China) Co., Ltd.; Agarose G-10 and Neutral Red, purchased from Shanghai Aladdin Biochemical Technology Co., Ltd.; bicinchoninic acid assay (BCA) Protein Assay Kit, neutral protease, trypsin, CCK-8, Interleukin 1β (IL-1β), and Tumor necrosis factor alpha (TNF-α) detection kits, purchased from Beyotime Biotechnology of Shanghai.

The present disclosure further separates and purifies the enzymatic hydrolysate of holothurian so as to obtain the components with high purity and strong activity; ultrafiltration and gel chromatography are the methods commonly used for separation and purification at present; mass spectrometry identification technology features simple operation, sensitive detection and accurate results, etc., and the amino acid sequence of the active peptide is obtained by comparing the mass spectrometry data with the database of the target proteins. Molecular docking is an electronic simulation process for screening and designing target efficient bioactive compounds, which is now widely used for high-throughput and virtual enzymatic screening, and for predicting the binding sites, energies, and interaction forces between ligands and receptors, with the advantages of short time-consumption and high efficiency. Toll-like receptor 2 (TLR-2) is located on the cell surface and is a cytokine with relatively broad ligand specificity; it recognizes pathogen-associated molecular patterns for defence against invading organisms and has emerged as a potent therapeutic target.

### Embodiment 1 Isolation and Purification of Active Peptides

According to the present disclosure, the holothurian enzymatic hydrolysate is used as a raw material, which is separated and purified by ultrafiltration and gel chromatography, and components with strong immune activity are screened by measuring the influence of each component on the cell survival rate, cell phagocytosis, NO release and cytokine (IL-1B, TNF-α) secretion of macrophage RAW264.7.

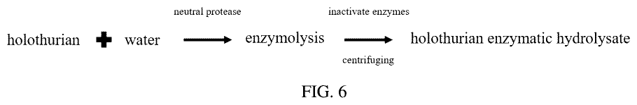

Enzymatic hydrolysis process: the material-liquid ratio of holothurian to water is 1:12 (mass/volume (m/v), grams per milliliter (g/mL)), the addition amount of neutral protease is 0.6%, the enzymolysis duration is 300 minutes (min), the temperature is 50 degrees Celsius (° C.), and the pH is 7.0. The enzymolysis is followed by water bath heating at 100° C. for 10 min to inactivate the enzymes, and centrifuging at 8000×g at 4ºC for two times to obtain the supernatant, and then the holothurian enzymatic hydrolysate is obtained (see FIG. 6).

### Method for Separation and Purification

(1) Ultrafiltration: studies have shown that peptides with low molecular weights are more active than those with high molecular weights, and that peptides with low molecular weights may be completely absorbed in the intestinal environment while maintaining their original physiological activities; accordingly, ultrafiltration centrifugation tubes of <3 kilodaltons (kDa) are selected to ultrafiltrate the enzymatic hydrolysate, and fractions of <3 kDa are obtained for subsequent activity studies.

(2) Gel chromatography: a SephadexG-15 chromatographic column filled with dextran gel resin is used; the sample concentration is 20 mg/mL; the injection volume is 2 mL; the flow rate is 1 mL/min; the ultraviolet detection wavelength is 214 nanometers (nm). Collections from the same peak are mixed, concentrated and freeze-dried. The most immunologically active fractions are screened by the following measuring indexes.

### Measuring Indexes

(1) Influence of enzymatic hydrolysate on the survival rate of macrophages RAW264.7

Blank group: macrophages RAW264.7 are inoculated into 96-well plates and cultured for 24 hours (h).

Sample group: normal cultured macrophages RAW 264.7 are treated with enzymatic hydrolysate of different concentrations (50-800 micrograms per milliliter (μg/mL) for 24 h.

CCK-8 method is used to determine the absorption values of different components.

(2) Influence of enzymatic hydrolysate on phagocytosis of macrophage RAW264.7

Blank group: macrophages RAW264.7 are inoculated into 96-well plates and cultured for 24 h.

Sample group: different concentrations (50-800 μg/mL) of enzymatic hydrolysate are used to treat Raw 264.7 for 24 h.

The absorbance values of different components are measured at 550 nm.

(3) Influence of enzymatic hydrolysate on the release of NO and cytokines (IL-1β, TNF-α) from macrophages RAW264.7

Blank group: Macrophage RAW264.7 cells are inoculated into a 96-well plate and cultured for 24 h.

Sample group: normal cultured cells are treated with different concentrations (50-800 μg/mL) of enzymatic hydrolysate for 24 h.

Positive control group: normal cultured cells are treated with 100 ng/ml LPS for 24 h.

Different components are determined by NO and enzyme-linked immuno sorbent assay (ELISA) kit.

The results show that the enzymatic hydrolysate is separated into two components at 214 nm by SephadexG-15 column, namely, SCH-N-I-1 and SCH-N-I-2. Under the addition amount of SCH-N-I-2 component of 200 μg/mL, the highest cell survival rate and the highest phagocytosis of absorbance response are 133.95±1.56% and 0.1117±0.0009, respectively, and the release of NO and cytokines (IL-1β, TNF-α) is high in the concentration range. The immune component SCH-N-I-2 with the strongest activity is selected for further study.

### Embodiment 2 Identification of Amino Acid Sequence of Active Peptide

LC-ESI-MS/MS is used to analyze the immune component SCH-N-I-2 purified by gel separation. By comparing with the holothurian protein database, 13 amino acid sequences of novel immune active peptides with high hydrophobicity and immune amino acids are screened (see Table 1).

### Embodiment 3 Screening of Active Peptides

The toxicity of 13 new immunocompetent peptides obtained in Embodiment 2 is predicted by online tool Toxinpred (https://webs.iiitd.edu.in/raghava/toxinpred/multi_submit.php), and the results are illustrated in Table 1.

TABLE 1
Biotoxicity prediction results of immunocompetent
peptides from holothurian and Libdock scoring table

| | | | Libdock score |
|---|---|---|---|
| | | | TLR-2 (PDB ID: |
| NO. | Peptide sequence | Biotoxicity | 1FYW) |
| 1 | IIENAVQ(SEQ ID NO. 1) | Non-Toxin | 155.88 |
| 2 | KTWFF(SEQ ID NO. 2) | Non-Toxin | 75.8533 |
| 3 | KSYELP(SEQ ID NO. 3) | Non-Toxin | 119.52 |
| 4 | KPRVGGR(SEQ ID NO. 4) | Non-Toxin | / |
| 5 | LSLFAQP(SEQ ID NO. 5) | Non-Toxin | / |
| 6 | LLDGQPR(SEQ ID NO. 6) | Non-Toxin | / |
| 7 | LFQPSFL(SEQ ID NO. 7) | Non-Toxin | 77.0673 |
| 8 | YCFNIPT(SEQ ID NO. 8) | Non-Toxin | / |
| 9 | QIRDVFN(SEQ ID NO. 9) | Non-Toxin | / |
| 10 | KDVGVHTR(SEQ ID NO. 10) | Non-Toxin | / |
| 11 | FAGDDAPRA(SEQ ID NO. 11) | Non-Toxin | 164.224 |
| 12 | VTCGVLKST(SEQ ID NO. 12) | Non-Toxin | / |
| 13 | LTERGYSF(SEQ ID NO. 13) | Non-Toxin | / |

### Embodiment 4 Molecular Docking Analysis of Active Peptides with TLR-2

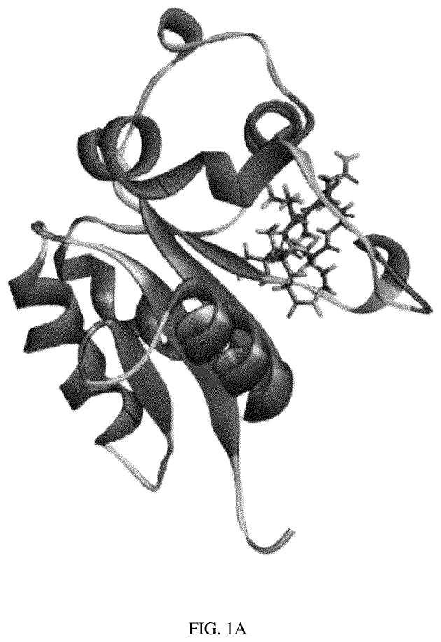

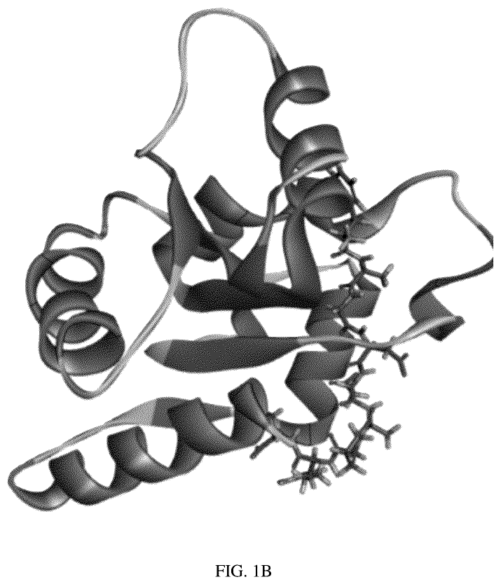

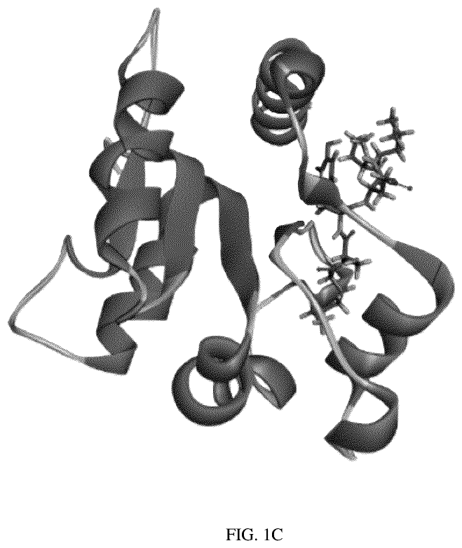

Using the software Discovery Studio 2017 R2, 13 immunocompetent peptides are molecularly docked with TLR-2 (PDB ID 1FYW) under the condition of CHARMm force field. By comparing Libdock Score, the more tightly docked immunocompetent peptides IIENAVQ, FAGDDAPRA and KSYELP are screened out, with results as shown in Table 1. The interactions between IIENAVQ(SEQ ID NO.1), FAGDDAPRA and KSYELP and TLR-2 are illustrated in FIG. 1A, FIG. 1B and FIG. 1C, respectively.

The interaction between active peptides and TLR-2 mainly includes van der Waals force, hydrogen bond, C-H bond, electrostatic interaction and alkyl/pi alkyl interaction. The amino acid residues of active peptides and TLR-2 catalytic site mainly include LEU734, ASP726, LYS751, TRP712, ALA731, ILE755, LYS759 and TYR761.

### Embodiment 5 Verification of Immune Activity of Active Peptides

Three screened immunocompetent peptides are synthesized by a solid phase method, and the purity of all of them is greater than 98%. Subsequently, the in vitro immune activities of the three immunocompetent peptides are verified.

### (1) Influence of Synthetic Peptide on the Cell Survival Rate of Macrophages RAW264.7

Blank group: Macrophages RAW264.7 are inoculated into 96-well plates and cultured for 24 h.

Sample group: synthetic peptides with different concentrations (50-200 μg/mL) are used for treating for 24 h.

The CCK-8 method is used to measure the absorbance values of different components, and the survival rate of macrophage RAW264.7 is calculated according to the absorbance values. The calculation formula is as follows:

$$Survival rate ( \% ) = \frac{OD Experimental group}{OD Control group} \times 100 \%$$

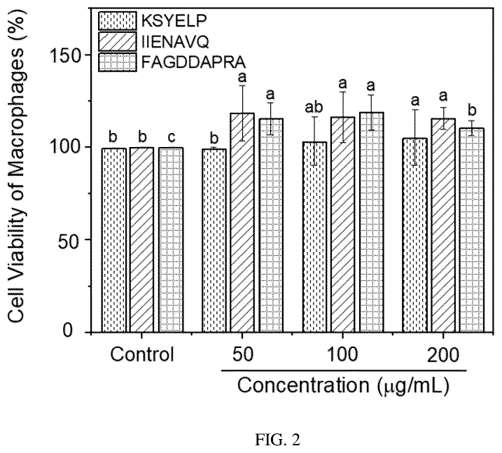

After 24 h of treatment with different concentrations of synthetic peptides, the survival rate of macrophage RAW264.7 is shown in FIG. 2.

(2) Influence of Synthetic Peptide on NO Release from Macrophage RAW264.7 Induced by LPS

Blank group: macrophage RAW264.7 cells are inoculated into a 96-well plate and cultured for 24 h.

Induced injury group: after cell culture, cells in blank group are treated with 100 ng/ml LPS for 24 h, and then treated with synthetic peptides with different concentrations (50-200 μg/mL) for 24 h.

Negative control group: after cell culture in blank group, cells are treated with 100 ng/mL LPS for 24 h.

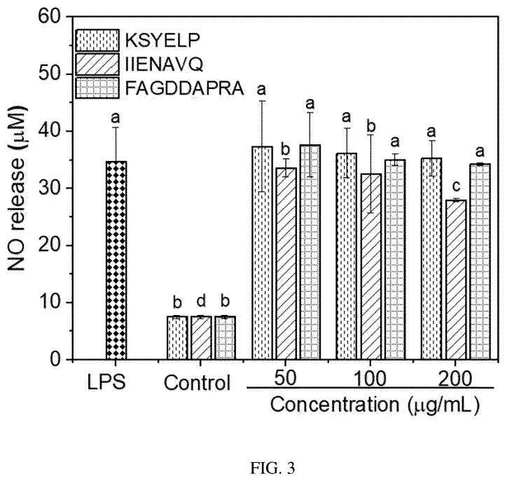

The NO release amount of different components is measured by NO kit. After 24 hours of treatment with synthetic peptides with different concentrations, the NO release amount of macrophage RAW264.7 induced by LPS is shown in FIG. 3.

The results show that among the three synthetic peptides in the concentration range of 50-200 μg/mL, IIENAVQ promotes cell growth most significantly, with a cell survival rate of 116.88%, and IIENAVQ significantly reduces the release of RAW264.7 NO from macrophages induced by LPS, compared with LPS-negative control group. IIENAVQ, the most active peptide discussed above, is selected for subsequent studies.

### Embodiment 6 Verification of the Influence of IIENAVQ on Cytokine Secretion

Influence of IIENAVQ activation and LPS-induced cytokines (IL-1β, TNF-α) secretion of macrophage RAW264.7

(1) The influence of cytokines (IL-1β, TNF-α) secretion of macrophage RAW264.7 activated by IIENAVQ.

Blank group: macrophage RAW264.7 cells are inoculated into a 96-well plate and cultured for 24 h.

Sample group: after cell culture in blank group, cells are treated with different concentrations (50-200 μg/mL) of IIENAVQ for 24 h.

Positive control group: after cell culture in blank group, cells are treated with 100 ng/mL LPS for 24 h.

(2) Influence of IIENAVQ on the secretion of cytokines (IL-1β, TNF-α) of macrophage RAW264.7 induced by LPS

Blank group: macrophage RAW264.7 cells are inoculated into a 96-well plate and cultured for 24 h.

Induced injury group: after the cells in the blank group are cultured, the cells are treated with 100 ng/mL LPS for 24 h, and then treated with different concentrations (50-200 μg/mL) of IIENAVQ for 24 h.

Negative control group: after cell culture in blank group, cells are treated with 100 ng/mL LPS for 24 h.

The cytokines of different components are determined by ELISA kit.

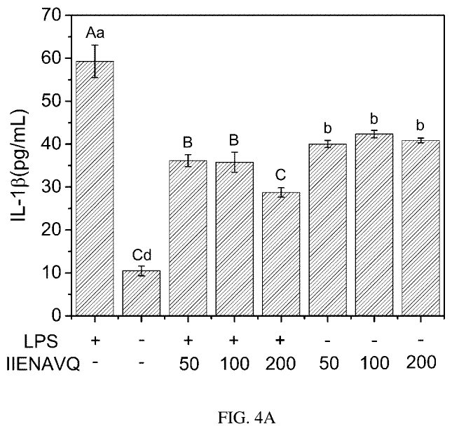

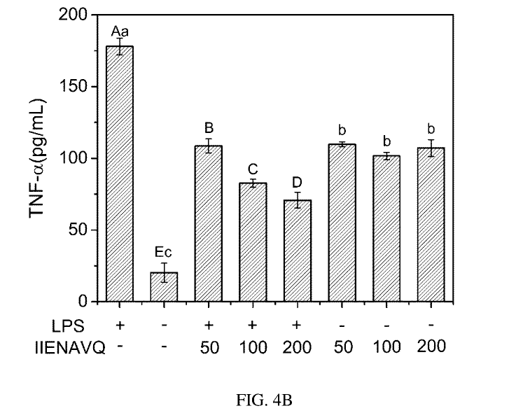

The final detection results of (1) and (2) are illustrated in FIG. 4A and FIG. 4B.

From the above results, it can be seen that the survival rate of RAW264.7 is 116.88% when the concentration of IIENAVQ is in the range of 50-200 μg/mL; when the concentration is 200 μg/mL, the release of NO is significantly reduced, and the final concentration of NO is 27.99±0.33 micromoles per liter (μmol/L). At the same time, the secretion of cytokines IL-1ß and TNF-α is also significantly reduced, which are 28.73±1.12 picograms per milliliter (pg/mL) and 70.78±5.55 pg/mL respectively, and the inhibition rates are 51.54 and 60.24% respectively. To sum up, the immune response is induced by IIENAVQ stimulation, but it is not over-activated, allowing the body to be maintained in a healthy and stable state.

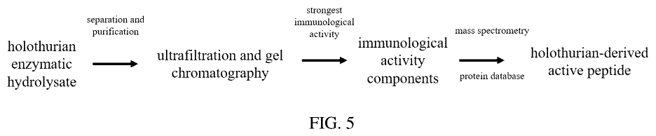

The preparation of holothurian-derived immunoactive peptides are illustrated in FIG. 5. as shown in the figure, the holothurian enzymatic hydrolysate is isolated and purified, and then the purified fractions are screened for immunoreactivity and matched with protein database to finally obtain the holothurian-derived immunoreactive peptides.

The above-mentioned embodiments only describe the preferred mode of the present disclosure, and do not limit the scope of the disclosure. Under the premise of not departing from the design spirit of the disclosure, various modifications and improvements made by ordinary technicians in the field to the technical scheme of the disclosure shall fall within the protection scope determined by the claims of the disclosure.

What is claimed is:

1. A holothurian-derived active peptide with immune activity, wherein an amino acid sequence of the holothurian-derived active peptide is shown in SEQ ID NO.1.

2. A preparation of immunocompetent peptide, comprising the holothurian-derived active peptide according to claim 1.

3. A preparation method of the holothurian-derived active peptide according to claim 1, comprising: using holothurian enzymatic hydrolysate as raw material, carrying out ultrafiltration and gel chromatography for separation and purification, screening out components of strongest immunological activity, and then identifying by secondary mass spectrometry, screening out the holothurian-derived active peptide by comparing with a holothurian protein database.

4. The preparation method according to claim 3, wherein a preparation method of the holothurian enzymatic hydrolysate comprises: mixing holothurian with water to obtain a mixture, adding neutral protease into the mixture for enzymolysis, carrying out water bath heating to inactivate enzymes at an end of the enzymolysis, and centrifuging twice, extracting a supernatant to obtain the holothurian enzymatic hydrolysate.

5. The preparation method according to claim 4, wherein a material-liquid ratio of the holothurian to water is 1 gram:12 milliliters.

6. The preparation method according to claim 4, wherein an addition amount of the neutral protease is 0.6% of a mass of the mixture, a duration of the enzymolysis is 300 minutes, a temperature is 50 degrees Celsius, and a pH is 7.0.

7. The preparation method according to claim 4, wherein a temperature of the water bath heating is 100 degrees Celsius and a duration is 10 minutes.

8. An application of the holothurian-derived active peptide according to claim 1 in preparing products for improving immunity.

9. The application according to claim 8, wherein the products comprise medicines.

10. An application of the preparation of immunocompetent peptide according to claim 2 in preparing products for improving immunity.

11. The application according to claim 10, wherein the products comprise medicines.


SEQUENCE LISTING (13 sequences)

SEQ ID NO: 1 - protein, 7 aa, synthetic construct
IIENAVQ

SEQ ID NO: 2 - protein, 5 aa, synthetic construct
KTWFF

SEQ ID NO: 3 - protein, 6 aa, synthetic construct
KSYELP

SEQ ID NO: 4 - protein, 7 aa, synthetic construct
KPRVGGR

SEQ ID NO: 5 - protein, 7 aa, synthetic construct
LSLFAQP

SEQ ID NO: 6 - protein, 7 aa, synthetic construct
LLDGQPR

SEQ ID NO: 7 - protein, 7 aa, synthetic construct
LFQPSFL

SEQ ID NO: 8 - protein, 7 aa, synthetic construct
YCFNIPT

SEQ ID NO: 9 - protein, 7 aa, synthetic construct
QIRDVFN

SEQ ID NO: 10 - protein, 8 aa, synthetic construct
KDVGVHTR

SEQ ID NO: 11 - protein, 9 aa, synthetic construct
FAGDDAPRA

SEQ ID NO: 12 - protein, 9 aa, synthetic construct
VTCGVLKST

SEQ ID NO: 13 - protein, 8 aa, synthetic construct
LTERGYSF